(19) United States
(12) Patent Application Publication
(10) Pub. No.: US 2026/0335226 A1
(43) Pub. Date: Oct. 29, 2026

(54) MUCOADHESIVEDRUGDELIVERYSYSTEMSANDMETHODSOFUSE

(71) Applicant: MADD OPHTHALMICS LTD., Vancouver (CA)

(72) Inventors: Andrew B. MERKUR, Vancouver (CA); Khoi Anh TRAN, Broadbeach (AU)

(21) Appl. No.: 19/114,397

(22) PCT Filed: Sep. 22, 2023
(86) PCT No.: PCT/CA2023/051260

§ 371 Date: Mar. 21, 2025

Related U.S. Application Data
(60) Provisional application No. 63/409,448, filed on Sep. 23, 2022.

(51) Int. Cl. A61K 9/00; A61K 45/06

(52) U.S. Cl. CPC A61K 9/0051; A61K 9/006; A61K 45/06

(57) ABSTRACT

This application relates to drug delivery systems comprising thin films configured for administration to the ocular mucosa. In some embodiments, a drug delivery system comprises a multi-layer thin film having a first bioadhesive layer for adhering to a mucosal surface at the administration site and a second non-adhesive layer coupled to the first layer, wherein at least one of the first layer and the second layer comprises at least one pharmaceutically active agent. In some embodiments the second layer limits dispersion of the at least one pharmaceutically active agent to enable focused delivery of a substantial portion of the at least one pharmaceutically active agent to a target location. In some embodiments the multi-layer film may comprise a third layer coupled to the second layer, wherein the first and third layers form external layers of the thin film and the second layer forms an internal layer of the thin film comprising the at least one pharmaceutically active agent. In some embodiments the drug delivery system may comprise packaging for storing the thin film in a sterile or semi-sterile state prior to ocular administration. The application also relates to methods employing thin films for delivering at least one pharmaceutically active agent to ocular locations in a subject. In some embodiments the mucoadhesive film is applied to the mucosal surface of the eye using an applicator.

### CROSS-REFERENCE TO RELATED APPLICATIONS

This application claims priority from U.S. Application No. 63/409,448 filed 23 Sep. 2022 and entitled MUCOADHESIVE DRUG DELIVERY SYSTEMS AND METHODS OF USE which is hereby incorporated herein by reference for all purposes. For purposes of the United States of America, this application claims the benefit under 35 U.S.C. § 119 of U.S. application No. 63/409,448 filed 23 Sep. 2022 and entitled MUCOADHESIVE DRUG DELIVERY SYSTEMS AND METHODS OF USE.

### TECHNICAL FIELD

This application relates to drug delivery systems comprising thin films configured for administration to the ocular mucosa. The application also relates to methods employing thin films for delivering at least one pharmaceutically active agent to ocular locations in a subject.

### BACKGROUND

All publications referred to herein are hereby incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Treatment of many ocular conditions or performance of ophthalmic procedures requires the topical application of drugs or other compositions to ocular tissue. For example, treatment of acute or chronic ocular conditions may require application of antibiotics, steroids, non-steroidal anti-inflammatory drugs (NSAIDs), or other medications to the eye. Further, ophthalmic procedures such as cataract surgery and intravitreal injections may require the delivery of anesthetics, antiseptics, emollients, mydriatics and/or other medicinal or non-medicinal agents to the eye, for example pre-procedure or post-procedure. The topical application of such compositions to the eye is often done using liquid drops. However, there are many drawbacks to the conventional approach. Often a large proportion of the drug or other active ingredient is wasted when the drops are not confined to the ocular administration site but rather drip on to a subject's cheek or drain into the subject's nasolacrimal duct. Such drainage may also result in unwanted systemic side effects in some cases. Eye drops often also pose other challenges including insufficient tissue contact time, contamination risks, storage and preservative requirements and relatively expensive shipping costs.

Often a series of eye drops (of different drugs) needs to be applied in a specific order prior to an ophthalmic procedure or examination. These repeat administrations are typically separated by extended wait periods. Such rounds of administration of distinct drugs adds significant cost, labour and complexity to an ophthalmic procedure. Patient compliance and the requirement of healthcare professionals to administer drops are significant drawbacks to conventional approaches.

The need has therefore arisen for systems and methods for reliably, conveniently, and economically delivering pharmaceutically active agents to a subject's eye in substitution for conventional drops. The need has particularly arisen for drug delivery systems employing thin films for reducing drug wastage, reducing wait time, increasing drug bioavailability or penetration, improving time-release of drugs, enhancing user comfort, reducing contamination risks and generally enabling more targeted delivery of optimum drug doses to the desired ocular locations.

Some intraocular films or pellets known in the prior art are directed towards subconjunctival or episcleral drug delivery to the eye. A number of these prior art references comprise bioerodible devices. An aim of some of the prior art is to deliver pharmaceuticals to the posterior region of the eye from a subconjunctival or episcleral site, as opposed to local delivery of pharmaceuticals to a subconjunctival, episcleral or other ocular administration sites. In addition, the prior art is limited by the configuration, shape and colour of the drug delivery systems.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides a drug delivery system comprising a thin film configured for positioning at an ocular administration site of a subject for delivery of at least one pharmaceutically active agent to a target location at or near the administration site. In some embodiments the thin film is configured to comprise: (a) a first bioadhesive layer for adhering to a mucosal surface at the administration site; and (b) a second layer coupled to the first layer, wherein at least one of the first layer and the second layer comprises the at least one pharmaceutically active agent.

Another aspect of the invention provides a method of delivering at least one pharmaceutically active agent to a target ocular location in a subject comprising providing a mucoadhesive film comprising the at least one pharmaceutically active agent; applying the film to a mucosal surface of an eye of the subject; and maintaining the film on the mucosal surface for a time period sufficient to enable delivery of the at least one pharmaceutically active agent to the ocular location.

Another aspect of the invention relates to use of a thin film to deliver at least one pharmaceutically active agent to an ocular administration site in a subject.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a cross-sectional side view of an embodiment of a drug delivery system of the present invention comprising a multi-layer mucoadhesive film.

FIG. 2 is a perspective view of the drug delivery system of FIG. 1.

FIG. 3 is a cross-sectional side view of the drug delivery system of FIGS. 1 and 2 contacting a mucosal surface.

FIG. 4 is a cross-sectional view of another embodiment of a drug delivery system of the present invention comprising a multi-layer mucoadhesive film.

FIG. 5 is a perspective view of the drug delivery system of FIG. 4.

FIG. 6 is a cross-sectional side view of the drug delivery system of FIGS. 3 and 4 contacting a mucosal surface.

FIG. 7 illustrates exemplary shapes of the mucoadhesive films of the present invention.

FIG. 8 is a perspective view of a drug delivery system comprising a packaging unit for supporting a mucoadhesive film.

FIG. 9 is a top plan view of a packaging unit for supporting a plurality of mucoadhesive films.

FIG. 10 illustrates exemplary ocular surfaces to which the drug delivery system of the present invention may be applied in regions of the eye and its substructures.

FIG. 11 is a top plan view of a packaging unit for supporting a plurality of mucoadhesive films wherein the films comprise different pharmaceutically active and/or non-medicinal agents.

FIG. 12 is a top plan view of a drug delivery system comprising a packaging unit for supporting a mucoadhesive film having a plurality of different portions comprising different pharmaceutically active and/or non-medicinal agents.

FIG. 13 is a top plan view depicting the film of FIG. 12.

FIG. 14 is a cross-sectional side view of the drug delivery system of FIG. 13.

FIG. 15 is a cross-sectional perspective view of the drug delivery system of FIG. 13.

FIG. 16 is a cross-sectional side view of a drug delivery system comprising a plurality of stacked, partially overlapping films.

FIG. 17 is a cross-sectional perspective view of the drug delivery system of FIG. 16.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

In some embodiments the present invention relates to a drug delivery system comprising a dosage form suitable for in vivo administration to an administration site. In some embodiments the dosage form is a thin film positionable at the administration site for delivering at least one pharmaceutically active agent to a target location. In some embodiments the administration site is an ocular site in a subject's eye and the target location is a location at or near the ocular site. For example, the target location may be a portion of the subject's eye in need of treatment and/or affected by an ophthalmic procedure. In some embodiments the subject may be a human being. In other embodiments the subject may be a non-human animal, for example in the case of veterinary applications. In some embodiments the thin film may be configured to adhere to a mucosal surface at the administration site. In some embodiments the drug delivery system may comprise either a single or multi-layer film. In one embodiment, the thin film may comprise a single layer configuration comprising one or more pharmaceutically active agents. In another embodiment, the film may comprise a multi-layer configuration including a plurality of layers (i.e. two, three, four, five or more layers). In some embodiments the multi-layer film may comprise a first bioadhesive layer for adhering to a mucosal surface at the administration site and a second and/or third non-adhesive layer coupled to the first layer, wherein at least one of the layers comprises the at least one pharmaceutically active agent.

The film may be formulated for rapid, sustained and/or extended release of the at least one pharmaceutically active agent. In some embodiments the at least one pharmaceutically active agent may comprise a plurality of different pharmaceutical agents. In some embodiments, the different pharmaceutical agents are delivered directionally, for example in a desired direction from the administration site toward a target location. In some embodiments, the different pharmaceutical agents are delivered sequentially (in relation to one another) to a target location. In some embodiments, the delivery of the different pharmaceutical agents is time-released. The at least one pharmaceutically active agent may be combined with other compounds, compositions or constituents which facilitate administration and use of the at least one pharmaceutical active agent but which do not necessarily have a medicinal effect. For example, the non-medicinal agents or constituents may comprise emollients, mydriatic agents, lipid compounds, or other inactive or inert constituents, such as diluents, carriers, excipients or combinations thereof. In some embodiments the present invention encompasses systems, compositions, methods and uses for delivering non-medicinal agents or constituents to a target location, optionally in combination with at least one pharmaceutically active agent or separately therefrom.

In some embodiments the dosage form may be formulated from one or more pharmaceutically-acceptable, water-soluble, film-forming polymers. As described herein, the polymers may be formed into a flexible thin film using, for example, a solvent casting or electrospinning technique. In some embodiments the polymers may be selected so that the at least one pharmaceutically active agent is bioabsorbable at the target location. For example, in some embodiments the polymers may form a bioabsorbable substrate that is impregnated or associated with the at least one pharmaceutically active agent and/or other non-medicinal agents or constituents formulated for delivery at the administration site. In some embodiments, the film is dissolvable. In some embodiments the dosage form comprises a hydrogel, including a hydrogel film.

The system may also comprise a packaging unit that is configured to store one or more thin films in a sterile or semi-sterile state prior to administration. In some embodiments the packaging unit enables rapid removal and administration of a thin film. In some embodiments the packaging unit may comprise a plurality of thin films arranged in a stacked or parallel array. In some embodiments the thin film may be removed from the packaging unit and deployed at an administration site with a separate instrument, such as a hand-held applicator.

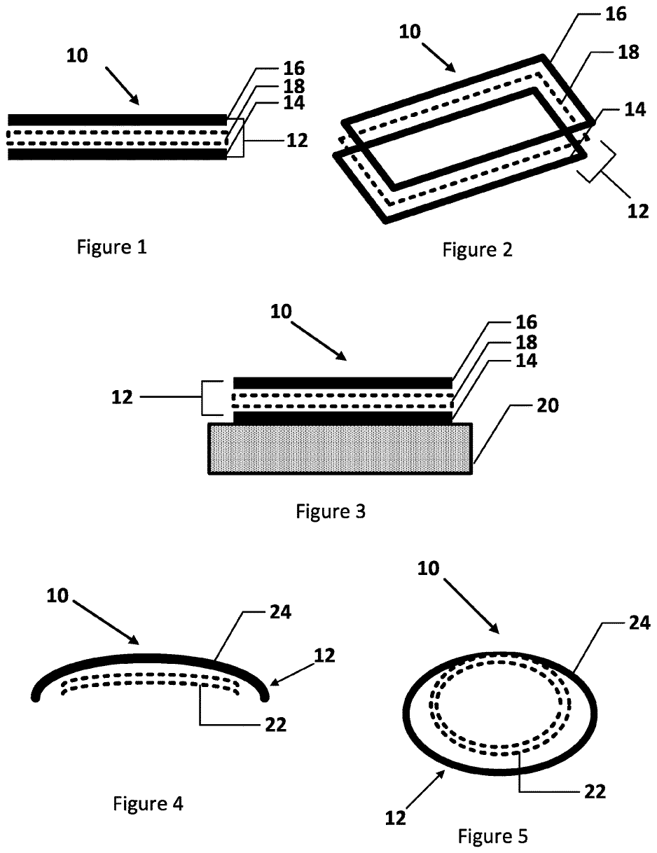

Referring to FIGS. 1 and 2, in one embodiment a drug delivery system 10 comprises a multi-layer thin film 12. In this embodiment, film 12 comprises a first bioadhesive layer 14, a second non-adhesive layer 16 and a third layer 18 disposed between layers 14 and 16. Each layer 14-18 may be formed from one or more water-soluble, film-forming, pharmaceutically acceptable polymers. Bioadhesive layer 14 may be configured to be placed into contact with a mucosal surface, such as an ocular mucosal surface. In addition to ophthalmic applications, the thin films of the invention may be useful in other applications for treating conditions, disorders or diseases relating to, for example, urology, gastroenterology, gynecology and ENT (Ears, Nose and Throat). Other possible mucosal surfaces therefore include nasal, pharyngeal, oral, vaginal, urethral and anorectal mucosal surfaces. Bioadhesive layer 14 may comprise bioadhesive polymers selected to interact with mucosal membranes for extended periods of time by interfacial forces. Non-adhesive layer 16 may form a backer structure of film 12 and third layer 18 may form an intermediate layer disposed between layers 14 and 16 in a “sandwich” configuration. At least one of the layers may comprise the at least one pharmaceutically active agent. Optionally some or all of the layers of a multi-layer film may comprise a pharmaceutically active agent. For example, in some embodiments bioadhesive layer 14 may comprise a pharmaceutically active agent. In some embodiments, layer 16 and/or layer 18 may be an adhesive layer, contain one or more pharmaceutically active agents, or be inert.

In some embodiments layers 14-18 may carry different pharmaceutically active agents or other non-medicinal agents or constituents. For example, in some embodiments the outer layers 14 and 16 of film 12 may comprise emollients and/or anesthetics and intermediate layer 18 may comprise at least one pharmaceutically active agent, for example for treatment of an ocular disease or condition. By way of example, ocular emollients may comprise artificial tears or other lanolin preparations and/or oleaginous ingredients and anesthetics may comprise one or more of tetracaine, alcaine or lidocaine. By way of further example, the at least one pharmaceutically active agent may comprise one or more of glaucoma drugs, anti-inflammatories, anti-histamines, antibacterials, antivirals, antifungals, antimetabolites, T-cell inhibitors, alkylating agents, biologic agents, azoles, fluorinated pyrimidines, adrenergics, anti-cholinergics, anesthetics, antiseptics, prostaglandins, carbonic anhydrase inhibitors, beta blockers, alpha-adrenergics and combinations thereof. In some particular embodiments, the pharmaceutically active or non-medicinal agents may include agents routinely included in clinical eye drops such as phenylephrine (adrenergic), mydriacyl (anti-cholinergic), lidocaine (amide anesthetic), tetracaine (ester anesthetics), CHX (aqueous chlorhexidine/antiseptics), povidone-iodine, emollients, and hyaluronic acid (artificial tears).

As will be understood by a person skilled in the art, a wide variety of pharmaceutically active agents may be used in ocular or non-ocular applications, either singly or in combination. Various non-limiting examples of pharmaceutically active agents are described herein. For example, anti-inflammatory include steroids such as one or more of difluprednate, loteprednol etabonate, prednisolone acetate, prednisolone sodium phosphate, rimexolone, fluorometholone acetate and fluorometholone alcohol, and non-steroidal anti-inflammatory drugs (NSAIDs) such as one or more of ketorolac tromethamine, bromfenac, nepafenac and diclofenac sodium. Anti-bacterials may include one or more of besifloxacin, ciprofloxacin, moxifloxacin, ofloxacin, gatifloxacin, tobramycin, gentamicin, polymyxin B, trimethoprim, gramicidin, neomycin, bacitracin, azithromycin and erythromycin. Antivirals may include one or more of acyclovir, ganciclovir, trifluridine and idoxuridine. Antifungals may include one or more of nystatin, natamycin, and amphotericin B. Azoles comprise one or more of ketoconazole, miconazole, fluconazole, itraconazole, econazole, clotrimazole, and voriconazole. Fluorinated pyrimidines include flucytosine. Antimetabolites may include one or more of methotrexate, mycophenolate mofetil (MMF), and azathioprine. Anti-histamines may include one or more of ketorolac tromethamine, ketotifen fumarate, loteprednol etabonate, bepotastine besilate, epinastine HCL, emedastine difumarate, alcaftadine, azelastine hydrochloride, olopatadine hydrochloride, nedocromil sodium, lodoxamide tromethamine and cromolyn sodium. T-cell inhibitors may include one or more of cyclosporine, tacrolimus, and sirolimus. Alkylating agents comprise one or more of cyclophosphamide and chlorambucil. Biologic agents may include one or more of tumor necrosis factor (TNF) inhibitors, lymphocyte inhibitors and interleukin inhibitors. Adrenergics may include phenylephrine. Anti-cholinergics comprise mydriacyl. Anesthetics may include one or more of lidocaine, alcaine and tetracaine. Antiseptics may include one or more of aqueous chlorhexidine (CHX) and povidone-iodine. Alpha-adrenergics may include one more of brimonidine and apraclonidine. Beta blockers include one or more of timolol, levobunolol and betaxolol. Carbonic anhydrase inhibitors may include one or more of brinzolamide and dorzolamide. Prostaglandins may include one or more of latanoprost, tafluprost, bimatoprost, and travoprost. As will be understood by a person skilled in the art, many other pharmaceutically active agents may be suitable for ocular or non-ocular administration in addition to the foregoing non-limiting examples.

As illustrated in FIG. 3, thin film 12 may be placed in contact with a mucosal surface 20 at an administration site. In particular, thin film 12 may adhere to mucosal surface 20 by bioadhesive layer 14 contacting and associating with mucosal surface 20. As explained above, in some embodiments the configuration of thin film 12 enables delivery of bioabsorbable elements comprising pharmaceuticals or pharmaceutically inactive constituents associated with first layer 14 and/or second layer 16 to mucosal surface 20 before the delivery of pharmaceuticals associated with film layer 18. For example, film 12 may be configured so that film layers 14 and 16 comprise anesthetics and/or emollients which are delivered to or near the administration site (e.g. to mucosal surface 20). After layers 14 and 16 have solubilized, dissolved, degraded and/or eroded at the administration site, the one or more pharmaceutically active agents of layer 18 may then contact the mucosal surface 20 and disperse to the target location, for example to confer a local or systemic therapeutic effect.

In some embodiments the thin film 12 may also be configured to deliver pharmaceutically active agents in a directional manner, for example in a unidirectional manner toward a target location. In some embodiments, the direction of flow of the one or more pharmaceutically active agents is from the layer of origin of the one or more pharmaceutically active agents (e.g. one or more layers 14-16) to the mucosal surface 20. In some embodiments, the outer layer (layer 16) causes the one or more pharmaceutically active agents to move unidirectionally toward the mucosal layer 20 and/or prevents exit of the one or more pharmaceutically active agents through layer 16. In some embodiments, the outer layer (layer 16) contains adhesive, one or more pharmaceutically active agents or is inert. In some embodiments, when the eyelid of the subject closes, layer 16 will come into contact with a mucosal surface opposite to or in the vicinity of mucosal surface 20, for example a mucosal surface of the subject's eyelid.

FIGS. 4 and 5 illustrate another embodiment of multi-layer film 12 comprising a first layer 22 and a second layer 24. In this embodiment, first layer 22 is a bioadhesive layer similar to bioadhesive layer 14 configured for placement in contact with a mucosal surface 20 and layer 24 is a second layer coupled to first layer 22 in a manner similar to layer 16. In this embodiment layer 22 may comprise at least one pharmaceutically active agent and layer 24 may comprise an inert, slow-dissolving pharmaceutically acceptable polymer which blocks or limits the dispersion of the at least one pharmaceutically active agent. In this specification, “slow-dissolving” refers to relatively slower kinetics of solubilisation, degradation or erosion of layer 24 relative to layer 22 based on parameters such as composition or thickness of the respective layers. In this embodiment layer 24 partially encapsulates layer 22 to help direct dispersion of the at least one pharmaceutically active agent toward the target location. This configuration thus focuses delivery of the pharmaceuticals or other constituents associated with bioadhesive layer 22 to the mucosal site of application as described above and minimizes unwanted dispersion of pharmaceuticals or other constituents to other regions of the eye, for example the tarsal conjunctiva rather than the bulbar conjunctiva. This may have the effect of enhancing drug bioavailability at the target location and reducing drug wastage and possible unwanted side effects.

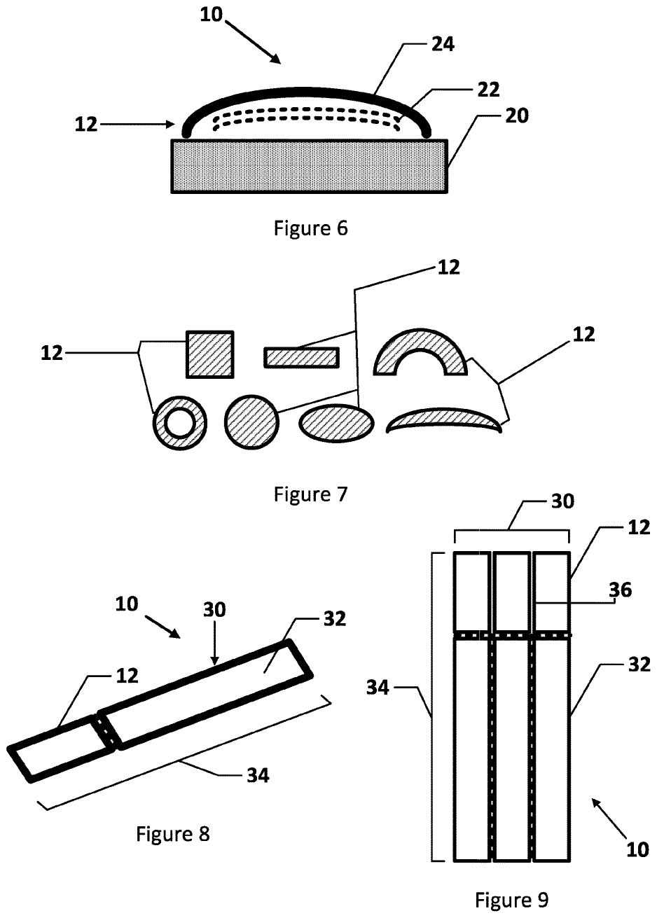

As shown in FIG. 6, in some embodiments the thin film of FIGS. 4 and 5 may contact a mucosal surface 20 through both first bioadhesive layer 22 and second layer 24. In this embodiment at least part of layer 24 (e.g. end portions thereof) could also optionally be bioadhesive.

Another aspect of the invention provides for one or more dyes, colourants or inspecting agents to be impregnated in or associated with any or all layers comprising thin film 12. Such embodiments may be directed towards temporarily marking the ocular administration site of thin film 12 and/or the in vivo location (e.g. dispersion) of pharmaceutically active agents or other constituents. Such dyes, colourants and inspecting agents may include, for example, fluorescein, gentian violet, trypan blue, brilliant blue, indocyanine green, and infracyanine green. These dyes, colourants and inspecting agents may assist in determining which drug or other pharmaceutically active agent is used, the location where the drug has been administered and/or the tissue(s) affected. In some embodiments, short duration dyes may be used to assess if the thin film is fully absorbed. In some embodiments, immediate-to long-acting dyes (e.g. 24 hrs) temporarily stain the site where the film is administered thereby aiding in visualizing the procedural or surgical site. In some embodiments, different colours are used to indicate the administration of different pharmaceutically active agents (e.g. red=dilation, blue=antiseptic, etc.).

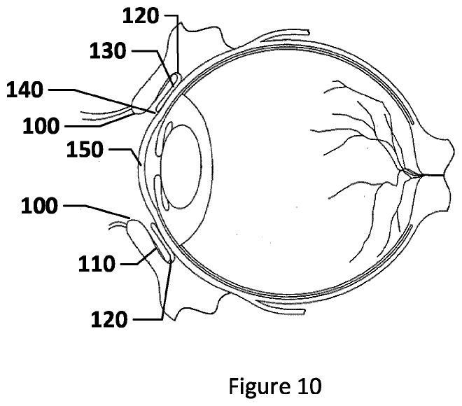

In order to facilitate positioning and use of drug delivery system 10, thin film 12 may be shaped and sized to conform the contour of an ocular surface at the site of administration. As will be apparent to a person skilled in the art, “ocular surface” refers to regions of the eye and its substructures (FIG. 10) including but not limited to the lid margin 100, tarsal conjunctiva 110, fornix 120, bulbar conjunctiva 130, corneoscleral limbus 140, cornea 150, or a combination thereof. By way of non-limiting examples, FIG. 7 illustrates various possible shapes of thin films 12 conforming to different ocular surfaces, such as square, rectangular, circular, ovular, annular and crescent shapes. Additional shapes that are similar to those demonstrated in FIG. 7 are also herein contemplated. In other embodiments, film 12 may include a footing or other structure for bearing film 12 against or otherwise seating film 12 relative to an ocular surface. By way of non-limiting examples, a circular, annular or crescent-shaped film 12 may be suitable for positioning at a corneal or corneoscleral location, and a rectangular, square or oval-shaped film 12 may be suitable for positioning at a juxta-tarsal location. Additional non-limiting examples include a large ring for positioning around the cornea at the limbus, and a rectangular shape that can be positioned at the fornix. Variability in the shape of the thin film and its configuration (e.g. layers, height, length, thickness) allow for the tailored administration of one or more pharmaceutically active agents in a particular procedure, examination or treatment.

In some embodiments, the administration of one or more pharmaceutically active agents is titratable. In some embodiments, the titration meets the clinical requirements of the procedure, examination or treatment through suitable shaping and/or configuration of the thin film. In another embodiment, the thin film is shaped for drug delivery to a specific location of the eye (e.g. superotemporal bulbar conjunctiva, corneal, corneoscleral, forniceal, tarsal plate and the like). In some embodiments, the shape and/or configuration of the thin film improves placement and speed of delivery of the one or more pharmaceutically active agents. Unlike conventional methods where eye drops may miss the target location of the eye or the timing of repeat drop installation is lengthy, in some embodiments the shape and/or configuration of the thin film provides for targeted, quick and accurate drug delivery.

Another aspect of the present invention provides for thin film 12 to deliver bioabsorbable elements contained therein in varying dosages and over varying durations. For example, variable dosages or durations may be modulated by varying the number and/or thickness of the layers (e.g. layers 14-16; layers 22-24) comprising thin film 12, the properties of the pharmaceutically acceptable polymers comprising the layers of film 12, and/or the concentration of the bioabsorbable elements such as the at least one pharmaceutically active agent and other non-medicinal or inactive agents associated with the layers comprising thin film 12. With respect to variable dosages, the bioabsorbable elements associated with thin film 12 are therefore titratable to treatment requirements. For example, thin film 12 may be configured to rapidly dissolve to allow for focused and localized delivery of an associated bioabsorbable element (e.g. to minimize dispersion), or configured to dissolve slowly to allow for release of a bioabsorbable element in a diffuse manner and over a longer duration.

Thin films of the present invention can be prepared by numerous methods known in the art. The selection and use of suitable film-forming mucoadhesive polymers would be readily apparent to a person skilled in the art having regard to prior publications in this field and common general knowledge: e.g. Karki et al., Thin films as an emerging platform for drug delivery, Asian Journal of Pharmaceutical Sciences, Volume 11, Issue 5, October 2016, pages 559-574; Park et al., Layer-by-layer assembled polymeric thin films as prospective drug delivery carriers: design and applications, Biomaterials Research, Volume 22, Article number: 29 (2018); Zelikin, Drug Releasing Polymer Thin Films: New Era of Surface-Mediated Drug Delivery, ACS Nano 2010, 4, 5, 2494-2509; Mucoadhesive Polymers: Strategies, Achievements and Future Challenges, Advanced Drug Delivery Reviews, Volume 57, Issue 11, 3 Nov. 2005, pages 1553-1730; The use of mucoadhesive polymers in ocular drug delivery, ibid, pages 1595-1639; Bibliometric and visualized analysis of ocular drug delivery from 2001 to 2020, Journal of Controlled Release, Volume 345, May 2022, pages 625-645.

By way of example, thin films for controlled drug delivery may be prepared using a polymer or combination of polymers, such as hydroxypropyl methylcellulose (HPLC), polyvinyl pyrrolidone (PVP), sodium carboxymethylcellulose (CMC), polyvinyl alcohol (PVA) and hyaluronic acid (HA). The film forming technique may, for example, be solvent casting or electrospinning. The ratio of the polymers may be selected to optimize mucoadhesive, residence time, flexibility or other properties of the films. The durability of the films may be enhanced by post-casting modifications, such as crosslinking. The flexibility of the films may be optimized by adding plasticizers, such as glycerol and polyethylene glycol. The release profile of the active ingredients may also be optimized. In some embodiments, multi-layer films may be formulated.

With reference to FIGS. 8 and 9, drug delivery system 10 may further comprise structures for supporting, storing and/or administering films 12. In some embodiments such structures may comprise packaging units 30 for receiving one or more films 12 prior to administration. For example, as shown in FIG. 8, thin film 12 may be attached to or associated with a packaging unit 30 comprising a substrate 32. For example, substrate 32 may comprise an inert paper or non-medicated mucoadhesive strip and a thin film 12 may be positioned at an end portion thereof (medicated tip) to form a composite medicated strip 34. Thus drug delivery system 10 is configured in this embodiment to facilitate storage of thin film 12 and/or deployment of film 12 at an administration site when desired. For example, a user can manually handle and deploy drug delivery system 10 by holding or otherwise engaging substrate 32 of medicated strip 34 and applying film 12 to a mucosal surface 20. In this example, thin film 12 separates from substrate 32 when placed on a mucosal surface, thereby applying film 12 without direct handling or manipulation thereof. In various embodiments, film 12 and substrate 32 may be packaged as an individual strip 34 or as multiple strips 34, and may be packaged in a sterile or semi-sterile format. Thus, in some embodiments, the drug delivery system and/or its packaging is in a sterile or semi-sterile format. In some embodiments, the drug delivery system and/or its packaging has improved storage. The drug delivery system and/or packaging, for example, is easier to store than conventional treatments (e.g. eye drops). In some embodiments, the drug delivery system and/or its packaging is stored without the need for refrigeration (e.g. at room temperature). Unlike conventional methods (e.g. eye drops) where repeated use of containers may introduce microbes, in some embodiments, the drug delivery system and/or its packaging of the invention is not susceptible to contamination. This provides for a reduction or elimination of preservatives and decontaminants in the drug delivery system and/or its packaging. It also reduces the costs of discarding product in the case where an applicator (e.g. eye dropper bottle) comes in contact with a patient's ocular surface or eyelash. In some embodiments, the drug delivery system and/or its packaging is light weight (e.g. containing little water) compared to conventional treatments (e.g. eye drops) and is therefore easier and less costly to transport.

With reference to FIG. 9, in another example embodiment of the present invention, drug delivery system 10 may comprise a series of medicated strips 34, each comprising a thin film 12 and substrate 32, applied to an inert backer sheet 36. In some embodiments medicated strip 34 comprising thin films 12 may be arranged on backer sheet 36 in a stacked or parallel array. In some embodiments each medicated strip 34 is separated from adjacent strips 34 by perforations. In use, individual medicated strips 34 are removed from backer sheet 36 by manually holding or otherwise engaging a substrate 32 and separating the selected medicated strip 34 from adjacent strips 34 along the perforation lines and removing the selected medicated strip 34 from the backer sheet 36. The selected medicated strip 34 can then be used as described above to apply thin film 12 at an administration site. As in the embodiments described above, in the example of FIG. 9 the medicated strips 34 may be packaged in a sterile or semi-sterile format to avoid or limit risk of contamination of film 12.

In embodiments where drug delivery system 10 comprises a plurality of medicated strips 34 arranged in an array, at least some strips 34 of the array may comprise different pharmaceutically active agents (e.g. medications) or non-medicinal agents than other strips 34 of the array. This may enable easy administration of multiple pharmaceutically active agents or other preparations in sequence or in combination. In one example embodiment, drug delivery system 10 may comprise a stacked array of multiple medicated strips 34 comprising (1) an anesthetic (e.g. lidocaine); (2) a mydriatic agent (pupil dilator); and (3) fluorescein/emollient.

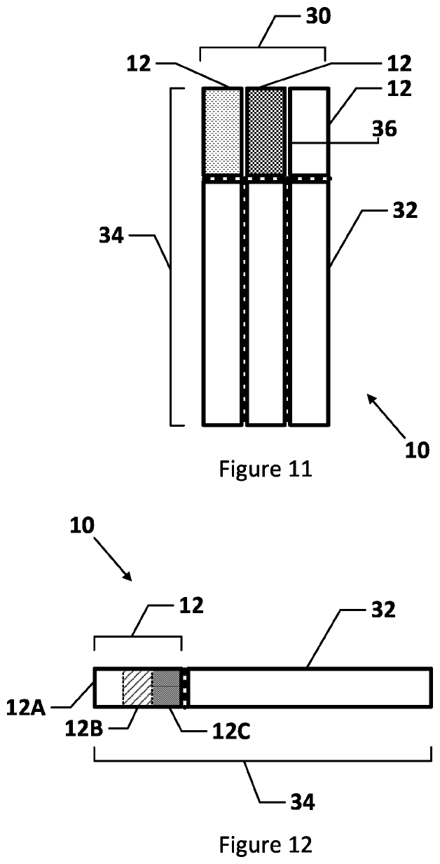

With reference to FIG. 11, drug delivery system 10 may comprise a packaging unit 30 for supporting a plurality of films 12 wherein the films 12 may comprise different pharmaceutically active and/or non-medicinal agents. The films 12 are arranged in a side-by-side array in this example. In this embodiment the strips 34 could be removed from the packaging unit sequentially and the respective films 12 could be administered sequentially as discussed above as part of a clinical or surgical procedure and/or ocular treatment. A packaging unit 30 may be configured so that strips 34 and the respective films 12 relate to a particular procedure and/or therapy. For example, a packaging unit 30 could comprise a one thin film 12 for use before an ophthalmic procedure (pre-procedure) and one thin film 12 for use after an ophthalmic procedure (post-procedure).

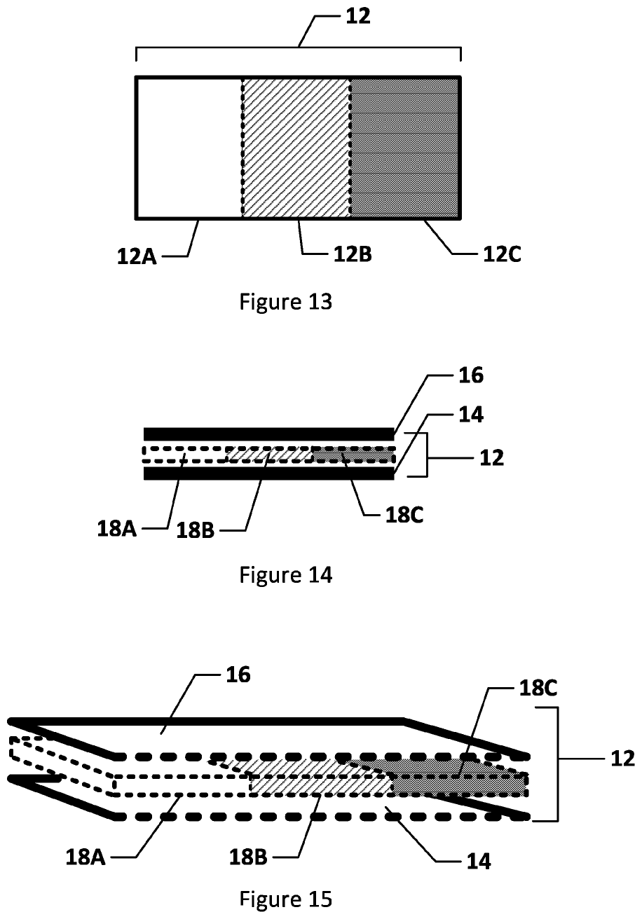

FIG. 12 illustrates an example embodiment of a drug delivery system 10 comprising a packaging unit 30 comprising a single medicated strip 34 supporting a thin film 12. In this embodiment film 12 may comprise a plurality of portions or sub-films 12A, 12B and 12C each optionally comprising different pharmaceutically active and/or non-medicinal agents. The portions 12A, 12B and 12C may be arranged side-by-side (FIGS. 12-15). In other embodiments portions 12A, 12B and 12C may partially or entirely overlap.

In some embodiments film 12 may be configured so that one or more layers 14-18 comprise a plurality of portions or sub-layers. For example, as shown in FIGS. 14 and 15, third layer 18 may comprise a plurality of portions or sub-layers 18A, 18B and 18C each optionally comprising different pharmaceutically active and/or non-medicinal agents. In the illustrated embodiment of FIGS. 14 and 15, portions 18A, 18B and 18C are arranged side-by-side. In other embodiments portions 18A, 18B and 18C may partially or entirely overlap, for example to form a tiered layer 18. In some embodiments each portion 18A, 18B and 18C may comprise different pharmaceutically active and/or non-medicinal agents configured for combined or sequential release at the site of administration.

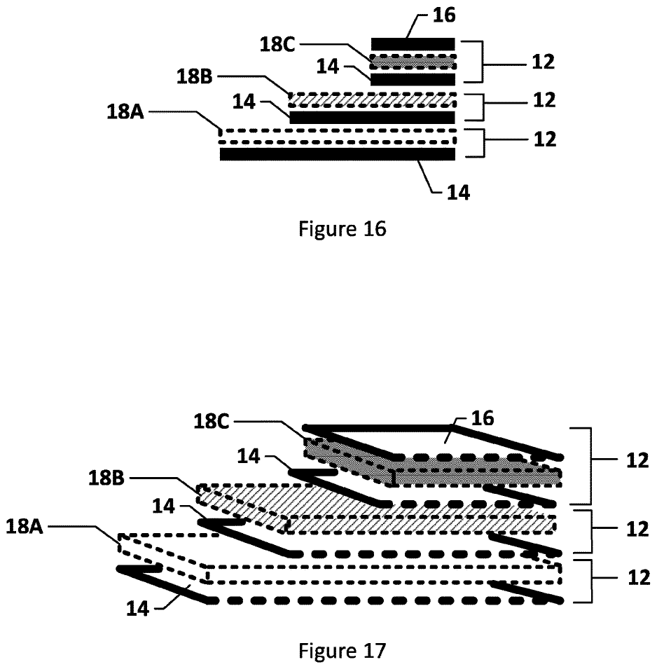

In some embodiments a drug delivery system 10 may comprise a plurality of stacked films 12 wherein the films 12 may each optionally comprise different pharmaceutically active and/or non-medicinal agents. For example, as shown in FIGS. 16 and 17, each film 12 may comprise a respective third layer 18 comprising a different portion 18A, 18B or 18C having a different size and/or make-up. In the embodiments of FIGS. 16 and 17, each thin film 12, including respective layers 18A, 18B and 18C, in the stacked array is of a different length and may comprise a correspondingly different amount of a pharmaceutically active and/or non-medicinal agent. In other embodiments some or all of the films 12 in the stacked array may be the same length. In the illustrated embodiment of FIGS. 16 and 17 layers 18A, 18B and 18C are separated by intervening layers 14 but other configurations are possible.

In some embodiments a drug delivery system 10, for example in the form of a thin film 12 or a medicated strip 34 comprising a film 12, may be deployed at the administration site with a separate instrument, such as a hand-held applicator. In some embodiments a disposable, moistened “Dabb” applicator could be used to engage film 12 and position it at the administration site. In another embodiment a forceps-like instrument could be employed to engage a portion of a strip 34 (e.g. a non-medicated portion of substrate 32) and position film 12 at an administration site.

In one embodiment of the invention, there is provided a method comprising the application of a thin film drug delivery system to an ocular surface for treatment of ocular conditions or as part of ophthalmic procedures, including pre-operatively and post-operatively. As described above, in some embodiments, the thin film is shaped to conform to an ocular surface to facilitate treatment of an ocular condition or as part of or to facilitate an ophthalmic procedure. In some embodiments the invention relates to a method of delivering at least one pharmaceutically active agent to a target ocular location in a subject comprising providing a mucoadhesive film comprising the at least one pharmaceutically active agent as described herein; applying the film to a mucosal surface of an eye of the subject; and maintaining the film on the mucosal surface for a time period sufficient to enable delivery of the at least one pharmaceutically active agent to the ocular location. In some cases, the thin film may be removed from the mucosal surface after the sufficient time period. In some examples the sufficient time period may be between approximately one minute and one hour. As discussed above, having a dye, colourant or inspecting agent may assist in removing the thin film when desired.

In other embodiments, the thin film and use thereof comprises particular methods of delivery of one or more pharmaceutically active agents. In some embodiments, the one or more pharmaceutical agents are delivered directionally from the thin film to the target location. In some embodiments, the one or more pharmaceutically active agents are delivered in sequence (in relation to one another) to the target location prior to a clinical or surgical procedure. Examples of drugs that may be delivered in sequence prior to a clinical procedure include delivery of an anesthetic agent followed by delivery of a mydriatic agent or agents. Examples of drugs that may be delivered in sequence prior to a surgical procedure include delivery of an anesthetic agent followed by an antiseptic agent. In some embodiments, the one or more pharmaceutically active agents are delivered in the treatment of an eye condition. Examples of drugs that may be delivered in sequence in the treatment of an eye condition include delivery of an anesthetic agent followed by delivery of an antibiotic, ocular antihypertensive and/or emollient. In some embodiments, the shape and configuration of the thin film provides controlled drug delivery. In some embodiments, the delivery of the one or more pharmaceutical agents is time-released. In one embodiment, at least one of the pharmaceutically active agents is rapidly released. In one embodiment, at least one of the pharmaceutically active agents is sustained released. In one embodiment, at least one of the pharmaceutically active agents is slow released. In one embodiment, at least one of the pharmaceutically active agents is rapidly released and sustained released. In some embodiments, the shape and configuration of the thin film is rapidly dissolvable for focused delivery of the one or more pharmaceutically active agents at a specific location. In some embodiments, the thin film provides for improved penetration of the one or more pharmaceutically active agents over conventional methods (e.g. eye drops). In some embodiments, the thin film provides for the administration of lower concentrations of the one or more pharmaceutically active agents compared to conventional methods. With improved penetration and/or reduced concentration of pharmaceutically active agents, in some embodiments, the patient may benefit from a reduction in side effects including, for example, a reduction in nasolacrimal drainage that may lead to systemic side effects.

In some embodiments, the thin films and methods of use include use for ophthalmic applications, including use in respect of ocular conditions and ophthalmic procedures. In this specification, “ocular condition” refers to acute and chronic diseases and conditions of the eye. Acute diseases and conditions include, but are not limited to blepharitis, allergic conjunctivitis, conjunctivitis (viral, bacterial, gonococcal, chlamydial), episcleritis, scleritis, corneal abrasion, recurrent corneal erosion syndrome, corneal ulcer, keratitis, acute anterior uveitis, iridocyclitis, intermediate uveitis, acute glaucoma and cystoid macular edema. Chronic diseases and conditions include, but are not limited to blepharitis, atopic conjunctivitis, vernal conjunctivitis, phlyctenular disease, neurotrophic corneal ulcer, persistent corneal epithelial defect, chronic uveitis, and glaucoma. In this specification, “ophthalmic procedures” refer to procedures including, but not limited to intravitreal injections, intracameral injection or paracentesis, cataract surgery, pterygium surgery and corneal procedures. Pre-operative applications of thin film 12 in ophthalmic procedures may include delivering anesthetics or antiseptics to the eye. Post-operative applications of thin film 12 in ophthalmic procedures may include delivering antibiotics, anti-inflammatories (e.g. non-steroidal anti-inflammatory drugs or steroids), ocular hypotensives, emollients, and mydriatics to the eye. In some embodiments, the thin films may be used for local ocular procedures including measurements of intraocular pressures, gonioscopy, contact lens insertions, removal of corneal and conjunctival foreign bodies, removal of conjunctival cysts and intravitreal or intracameral injections. In some embodiments the thin film is administered to the superotemporal bulbar conjunctiva. In other embodiments, the thin film is administered to the corneal, corneoscleral, forniceal, tarsal plate and the like.

In some embodiments the thin film is administered in a particular size. In some embodiments the thin film is 2×2 mm in size. In some embodiments the thin film is 4×4 mm in size. Other suitable sizes are also herein contemplated.

In some embodiments, the thin film contains at least one pharmaceutically active agent for administration, such as an anesthetic drug. In some embodiments the active agent is lidocaine. In some embodiments the lidocaine is present in the thin film in an amount of about 0.10 mg to about 0.50 mg, for example in an amount of about 0.12 mg to about 0.48 mg. Other suitable doses are also herein contemplated.

In some embodiments, the thin film provides a drug effect (e.g. anesthesia). In some embodiments, the drug effect of the thin film has a longer duration of effect as compared to eye drops. In some embodiments, the drug effect as delivered by thin film is longer than the drug effect as delivered by drops containing the same dose of the same drug. In some embodiments, the drug effect as delivered by thin film is longer than the drug effect as delivered by drops containing a higher dose of the same drug. In some embodiments, the thin film is capable of delivering at least 1, 2, 3, 4, 5, 10, 20, 30 or 60 minutes of drug effect (e.g. anesthesia). In some embodiments, administration of the thin film to a subject achieves better drug effect than drops containing the same or more amount of the pharmaceutically active agent. In some embodiments, administration of the thin film provides localised drug effect resulting in more accurate dosing and less side effects than administration with drops. In some embodiments, the thin film dissolves within 1 to 60 seconds following application to the ocular location. In some embodiments, the thin film dissolves within 5 to 50 seconds following application to the ocular location. In some embodiments, the thin film dissolves within 10 to 40 seconds following application to the ocular location. In some embodiments, the thin film dissolves within 15 to 25 seconds following application to the ocular location. In some embodiments, the thin film dissolves within 15 to 20 seconds following application to the ocular location.

As indicated above, use of the thin films and methods of use in respect of ocular conditions and ophthalmic procedures includes clinical and surgical use. Non-limiting examples of clinical use include eye examination and/or injections which may include administration of anesthesia, antiseptics, and/or mydriatics. In some embodiments, the thin film and methods of use are for surgical use. Non-limiting examples of surgical use include 1) preoperative use which may include the administration of anesthesia and antiseptics and/or mydriatics, for example, in procedures such as intravitreal injections (pneumatic, medication injections), intracameral (injection, paracentesis), cataract surgery, pterygium surgery, corneal procedures, and 2) postoperative use which may include the administration of antibiotics, anti-inflammatories (NSAIDS/steroids), ocular hypotensives, emollients/tear replacements, and/or mydriatics. In some embodiments, the thin film and methods of use are for treating a condition, disorder, or disease. In some embodiments, the condition, disorder or disease is blepharitis flare-up, seasonal allergic conjunctivitis, conjunctivitis (e.g. viral, bacterial, atypical: gonococcal, chlamydial), episcleritis/scleritis, corneal abrasion/recurrent corneal erosion syndrome, corneal ulcer, keratitis, acute anterior uveitis, iridocyclitis, intermediate uveitis, acute glaucoma (open and closed angle), cystoid macular edema, blepharitis-microbiome changes, atopic conjunctivitis/vernal conjunctivitis, phlyctenular disease, neurotrophic corneal ulcer, persistent corneal epithelial defect, chronic uveitis: anti-inflammatory, and primary open angle glaucoma/chronic angle closure glaucoma. A non-exhaustive list of pharmaceutically active agents for use in the thin film and methods of the invention include: 1) routine clinical eye drops comprising pharmaceutically active agents (optionally in combination with non-medicinal agents) such as phenylephrine (adrenergic), tropicamide (anti-cholinergic), cyclopentolate (anti-muscarinic), lidocaine (amide anesthetic), tetracaine (ester anesthetics), CHX (aqueous chlorhexidine/antiseptics), povidone-iodine, emollients, hyaluronic acid (artificial tears); 2) glaucoma drugs such as alpha-adrenergic (brimonidine, apraclonidine), beta-blockers specific and non-specific (timolol, levobunolol, betaxolol), carbonic anhydrase inhibitors (brinzolamide, dorzolamide), prostaglandins (latanoprost, tafluprost, bimatoprost, travoprost); 3) anti-inflammatories such as steroids (difluprednate, loteprednol etabonate, prednisolone acetate or sodium, phosphate, rimexolone, fluorometholone acetate or alcohol) and/or NSAIDS (ketorolac, bromfenac, nepafenac, diclofenac); 4) anti-histamines such as ketorolac tromethamine, ketotifen fumarate, loteprednol etabonate, bepotastine besilate, epinastine HCL, emedastine difumarate, alcaftadine, azelastine hydrochloride, olopatadine hydrochloride, nedocromil sodium, lodoxamide tromethamine, cromolyn sodium; 5) antibacterials such as besifloxacin, ciprofloxacin moxifloxacin, ofloxacin, moxifloxacin, gatifloxacin, tobramycin, gentamicin, polymyxinB/trimethoprim, polymyxinB/bacitracin, polymyxinB/neomycin/gramicidin, polymyxinB/neomycin/bacitracin, azithromycin, erythromycin, bacitracin; 6) antimetabolites such as methotrexate, mycophenolate mofetil, azathioprine; 7) T-cell inhibitors such as cyclosporine, tacrolimus, sirolimus; 8) Alkylating agents such as cyclophosphamide, chlorambucil; 9) biologic agents such as TNF inhibitors, lymphocyte inhibitors, interleukin inhibitors; 10) antivirals such as acyclovir, ganciclovir, TFT (topical trifluridine), topical idoxuridine; 11) antifungals such as nystatin, natamycin, amphotericin B; 12) azoles such as ketoconazole, miconazole, fluconazole, itraconazole, econazole, clotrimazole, voriconazole; and 13) fluorinated pyrimidines such as flucytosine. In some particular embodiments the pharmaceutically active agents include anti-VEGF agents (bevacizumab, ranibizumab, aflibercept, brolucizumab, faricimab), angiopoietin-2 and pilocarpine compounds.

### EXAMPLES

The following examples are intended to illustrate example embodiments of the invention but do not limit the scope of the invention in any way.

### Example 1—Use of Mucoadhesive Thin Films for Targeted Ocular Delivery of Anesthetic

Mucoadhesive single layer thin films were used for targeted ocular delivery of the anesthetic lidocaine. The thin films were compared to conventional eye drops comprising lidocaine. The thin films comprised water-soluble polysaccharide polymer, glycerol, lidocaine hydrochloride, and sodium bicarbonate. The conventional eye drops used in this example were Xylocaine 2% parenteral solution (AstraZeneca) available in plastic ampules (Polyamp® Duofit®) and administered as a single topical drop.

### Methods

The thin films were formed in 2×2 mm square strips containing 0.12 mg lidocaine. The thin films were placed on the superotemporal bulbar conjunctiva in four subjects. A drop of 2% lidocaine was placed in the contralateral eye. The average drop contained approximately 50 uL lidocaine solution or a dose of approximately 1 mg.

All thin films remained on the superotemporal bulbar conjunctiva until dissolved. The thin films were assessed for anesthetic effect at the administration site quadrant compared to other ocular quadrants (superonasal, inferonasal, inferotemporal) at time intervals of 15 seconds, 1 minute and 5 minutes. A cotton-tipped applicator was used to compare sensation in each quadrant. Sensation was measured as normal (N), reduced (R) or absent (A).

### Observations

The following sensations listed in Table 1 were observed after 15 seconds in respect of the subject eye receiving the thin film.

TABLE 1

| | Superotemporal | Superonasal | Inferonasal | Inferotemporal |
|---|---|---|---|---|
| Subject 1 | A | N | R | A |
| Subject 2 | A | N | R | A |
| Subject 3 | A | N | R | R |
| Subject 4 | A | R | R | A |

The following sensations listed in Table 2 were observed after 1 minute in respect of the subject eye receiving the thin film.

TABLE 2

| | Superotemporal | Superonasal | Inferonasal | Inferotemporal |
|---|---|---|---|---|
| Subject 1 | A | R | R | A |
| Subject 2 | A | R | A | A |
| Subject 3 | A | R | R | R |
| Subject 4 | A | R | R | A |

The following sensations listed in Table 3 were observed after 5 minutes in respect of the subject eye receiving the thin film.

TABLE 3

| | Superotemporal | Superonasal | Inferonasal | Inferotemporal |
|---|---|---|---|---|
| Subject 1 | A | R | A | A |
| Subject 2 | A | A | A | A |
| Subject 3 | A | A | A | A |
| Subject 4 | A | R | A | A |

By comparison, at all time points the lidocaine drops administered to the contralateral eye induced anesthesia with absent (A) responses in all 4 quadrants. Two of the four subjects had medication overflow onto the subject's cheek.

In respect of the subject eyes receiving the thin films, qualitative anesthesia occurred most rapidly superotemporally at the site applied followed by the inferotemporal, inferonasal and superonasal quadrants. This demonstrates the ability to localize the effects of an active ingredient (lidocaine in this example) using mucoadhesive thin films. This also demonstrates that mucoadhesive thin films provide a more efficient drug delivery system for targeted delivery of an active ingredient (lidocaine in this example), requiring less active ingredient to adequately anesthetize ocular surfaces for procedures when compared to drop solutions. Moreover, thin films allow for more accurate dose of active ingredients. Topical drops typically vary in volume (20-70 μL). As discussed in paragraph [0004] above, topical drops frequently overwhelm the ocular surface with leakage onto the face (wasted drug) with excess drops entering the nasolacrimal drainage system (risk for systemic side effects). An overall reduction in active drug dose would reduce the risks of systemic drug side effects.

### Example 2—Comparison Study of Use of Thin Films for Targeted Ocular Delivery of Anesthetic Having Extended Duration of Action

Single layer thin films as described in Example 1 above were used for targeted ocular delivery of the anesthetic lidocaine. The mucoadhesive thin films were compared to conventional eye drops. In this example, the conventional eye drops used were lidocaine 4% preservative free (Minims Lignocaine 4%, Bausch & Lomb, Macquarie Park NSW 2113, Australia) administered as a single topical drop. The comparison studies were designed to assess the safety, efficacy, onset, duration and preferences of the thin film against the current eye drop standard. The average conventional eye drop contained approximately 50 uL lidocaine solution or a dose of approximately 2 mg. A single drop of the conventional eye drops containing approximately 2 mg of lidocaine was instilled in the right eye of each subject while a small 4×4 mm square of mucoadhesive thin film containing 0.48 mg of lidocaine was placed in the inferonasal bulbar conjunctiva of each subject's contralateral left eye.

### Methods

Ten subjects participated in the study where each subject was administered a thin film or eye drop lidocaine formulation to each eye. Subjects were then asked to rate their levels of initial discomfort, time to resolution of discomfort, the quality of anesthesia and other additional comments. Clinical observations of the film and the eye as well as measurements were also performed. The testing was performed indoors at 23 degrees Celsius and humidity levels between 35% and 45%.

The thin film strips were delicately cut in to 4×4 mm squares (0.48 mg) and immediately instilled on to the conjunctival surface of each subject. Instillation was performed by an expert medical doctor on to the inferotemporal bulbar and tarsal conjunctiva. At the same time, a single drop of lidocaine 4% was instilled into the contralateral eye as described above. Each subject was monitored for 30 minutes following administration of the lidocaine.

Qualitative anesthetic testing was performed, using a cotton tip on the limbus, at predetermined intervals—baseline, 1 minute, 3 minutes, 5 minutes, 10 minutes, 30 minutes. Sensation was tested using cotton tip lightly palpating the inferotemporal corneoscleral limbus of each eye to assess sensation and symmetry. Patients were asked if they found any level of discomfort and whether there was a difference between the two lidocaine formulations when the cotton tip was applied against each eye. Observations performed by the examiner included noting the time to dissolution and disappearance of the film, testing of anesthesia of each eye at the predetermined time intervals and examination for any local irritation or reactivity on the surface of each eye. Slit lamp examination of each subject's eyes was also performed at baseline, prior to any testing and at the end of the 30 minutes. Additionally, the conjunctiva was inspected for any hyperemia, injection or abnormalities throughout the entire testing period following instillation of the mucoadhesive film and drops.

### Observations

The following observations set forth in Table 4 were made in terms of discomfort.

TABLE 4

| | Discomfort | | Resolution of | Dissolution |
|---|---|---|---|---|
| | Level | Discomfort | film discomfort | of film |
| | of film | Preference | (seconds) | (seconds) |
| Subject 1 | 4 | Drop | +20 | 25 |
| Subject 2 | 6 | Drop | +15 | 15 |
| Subject 3 | 6 | Same | NA | 12 |
| Subject 4 | 4 | Drop | +17 | 16 |
| Subject 5 | 4 | Drop | +8 | 15 |
| Subject 6 | 5 | Same | NA | 20 |
| Subject 7 | 7 | Same | +20 | 17 |
| Subject 8 | 7 | Drop | +12 | 16 |
| Subject 9 | 4 | Same | NA | 15 |
| Subject 10 | 5 | Drop | NA | 19 |

The following observations set forth in Table 5 were made in terms of anesthesia at different time intervals.

TABLE 5

| | Baseline | 1 m | 3 m | 5 m | 10 m | 30 m |
|---|---|---|---|---|---|---|
| Subject 1 | Same | Drop | Same | Same | Film | Film |
| Subject 2 | Same | Drop | Film | Film | Film | Film |
| Subject 3 | Same | Same | Same | Same | Same | Film |
| Subject 4 | Same | Drop | Same | Same | Same | Film |
| Subject 5 | Same | Same | Same | Same | Film | Film |
| Subject 6 | Same | Drop | Same | Same | Film | Film |
| Subject 7 | Same | Drop | Same | Same | Film | Film |
| Subject 8 | Same | Same | Same | Same | Film | Film |
| Subject 9 | Same | Same | Same | Same | Film | Film |
| Subject 10 | Same | Same | Same | Same | Same | Film |

The following further observations set forth in Table 6 were made in terms of discomfort, ocular appearance at site of administration and user preferences.

TABLE 6

| | Subject comments | | Preferences for |
|---|---|---|---|
| | respecting film | Appearance | Procedures |
| Subject 1 | Sting+ but stronger | Normal-no | Film |
| | | change | |
| Subject 2 | Longer, lid numb | Normal-no | Film |
| | | change | |
| Subject 3 | Lid numb | Normal-no | Film |
| | | change | |
| Subject 4 | Sting+, longer, lid | Normal-no | Film |
| | numb | change | |
| Subject 5 | Sting+, longer, lid | Normal-no | Film |
| | numb | change | |
| Subject 6 | Stronger, longer | Normal-no | Film |
| | | change | |
| Subject 7 | Stronger, longer | Normal-no | Film |
| | | change | |
| Subject 8 | Stronger, longer | Normal-no | Film |
| | | change | |
| Subject 9 | Stronger | Normal-no | Film |
| | | change | |
| Subject 10 | Longer | Normal-no | Film |
| | | change | |

### Results

There were no safety concerns from any of the subjects involved in the example study. There was clearly a longer duration of action of the thin films over the drops in all ten subjects. The extended duration of action of the film was a surprising result given that the dose of the lidocaine active ingredient was less. The initial discomfort and sting of the medications was subjectively equivalent in 4 of the cases while the remaining 6 preferred the drops in regard to the comfort of initial application as the stinging was less. Additionally, the discomfort of the film lasted longer in 6 of the subjects, but was minimal at an average extra duration of approximately 9 seconds over the entire cohort.

Despite the extra initial discomfort reported for the thin films, there was no evidence of conjunctival injection or hyperemia as evidence of hypersensitivity or inflammation. Subjects who noticed a longer resolution of discomfort also noted preference to anesthesia provided by the lidocaine drop, likely due to the extended time to discomfort resolution of the film. However by 3 minutes, the film showed equivalent subjective anesthesia in all cases, by 10 minutes the film superseded the drops with 7 of 10 subjects reporting better anesthetic effect with the film, and by 30 minutes the film demonstrated ongoing anesthetic action in all 10 subjects, whereas the drops had worn off. Additionally, some patients reported numbing of their periorbita and adnexa, including eyelids for periods beyond 60 minutes.

The results of this example demonstrate that the qualitative anesthetic efficacy of the thin film is equivalent by 3 minutes compared to lidocaine 4% drops and lasts beyond 30 minutes, longer in all cases compared to the drops.

Though the initial discomfort of the film lasted on average approximately 9 seconds longer, all patients tolerated the discomfort of the film and noted no issues with adverse outcomes both locally and systemically. Upon questioning which numbing agent would be preferred for ocular procedures including intraocular injections, all subjects in the cohort preferred the film due to its equivalent anesthesia effect but longer duration of action.

This example demonstrates that thin films comprising lidocaine are safe and effective in providing strong local anesthesia with extended duration of action. Accordingly, thin films may be considered for local ocular procedures including measurements of intraocular pressures, gonioscopy, contact lens insertions, removal of corneal and conjunctival foreign bodies, removal of conjunctival cysts and intravitreal or intracameral injections.

### INTERPRETATION OF TERMS

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed at different times

In addition, while elements are at times shown as being performed sequentially, they may instead be performed simultaneously or in different sequences. It is therefore intended that the following claims are interpreted to include all such variations as are within their intended scope.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

Various features are described herein as being present in “some embodiments”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

1-29. (canceled)

30. A method of delivering at least one pharmaceutically active agent to an ocular target location in a subject comprising:
(a) providing a mucoadhesive film comprising the at least one pharmaceutically active agent;
(b) applying the film to a mucosal surface of an eye of the subject; and
(c) maintaining the film on the mucosal surface for a time period sufficient to enable delivery of the at least one pharmaceutically active agent to the ocular target location.

31. The method of claim 30, further comprising removing the film from the mucosal surface after the time period.

32. The method of claim 30, wherein providing a mucoadhesive film comprises providing a packaging unit comprising the mucoadhesive film and removing the mucoadhesive film from the packaging unit, optionally, the packaging unit 1) is tearable to expose the mucoadhesive film, 2) maintains the film in a sterile or semi-sterile form, 3) is configured for single use or 4) comprises a plurality of mucoadhesive films which are each individually removable from the packaging unit and, optionally, the film is sealed within the interior of the packaging unit.

33. The method of claim 30, wherein the film is applied to the mucosal surface at or proximal to the ocular target location.

34. The method of claim 30, wherein the film is applied to the mucosal surface distal to the ocular target location and the at least one pharmaceutically active agent disperses to the ocular target location.

35. (canceled)

36. (canceled)

37. (canceled)

38. (canceled)

39. (canceled)

40. (canceled)

41. (canceled)

42. The method of claim 30, wherein the film comprises a bioabsorbable or bioerodible element for controllably dispersing the at least one pharmaceutically active agent at the target location.

43. The method of claim 30, wherein the at least one pharmaceutically active agent comprises a plurality of pharmaceutically active agents.

44. The method of claim 30, wherein the film is shaped to conform to the shape of the mucosal surface of the eye at or near the ocular location.

45. The method of claim 44, wherein the mucosal surface of the eye is a surface located in one or more of the lid margin, tarsal conjunctiva, fornix, bulbar conjunctiva (e.g. superotemporal bulbar conjunctiva), corneoscleral limbus, or cornea of the eye.

46. The method of claim 30, wherein the at least one pharmaceutically active agent is selected from the group consisting of glaucoma drugs, anti-inflammatories, anti-histamines, anti-bacterials, antivirals, antifungals, antimetabolites, anti-histamines, T-cell inhibitors, alkylating agents, biologic agents, azoles, fluorinated pyrimidines, adrenergics, anti-cholinergics, anesthetics (e.g. lidocaine), antiseptics, prostaglandins, carbonic anhydrase inhibitors, beta blockers, alpha-adrenergics and combinations thereof.

47. The method of claim 30 to 46, wherein the at least one pharmaceutically active agent is formulated to treat an ocular condition, wherein the ocular condition is one or more of blepharitis, conjunctivitis, episcleritis, scleritis, corneal abrasion, recurrent corneal erosion syndrome, corneal ulcer, keratitis, acute anterior uveitis, iridocyclitis, uveitis, glaucoma, cystoid macular edema, phlyctenular disease, neurotrophic corneal ulcer, persistent corneal epithelial defect.

48. The method of claim 30, wherein the at least one pharmaceutically active agent is formulated to facilitate an ophthalmic procedure, wherein the procedure is one or more of preoperative administration of anesthesia, antiseptics and/or mydriatics to facilitate intravitreal injections (pneumatic, medication injections), intracameral (injection, paracentesis), cataract surgery, pterygium surgery, corneal procedures, and/or wherein the procedure is one or more of postoperative administration of antibiotics, anti-inflammatories (e.g. NSAIDS/steroids), ocular hypotensives, emollients/tear replacements, and/or mydriatics.

49. (canceled)

50. The method of claim 30, wherein the film comprises a multi-layer film comprising: a first bioadhesive layer for adhering to a mucosal surface at the administration site; and a second layer coupled to the first layer, wherein at least one of the first layer and the second layer comprises the at least one pharmaceutically active agent and wherein, optionally, the second layer is non-bioadhesive.

51. (canceled)

52. The method of claim 50, wherein the second layer limits dispersion of the at least one pharmaceutically active agent to enable focused delivery of a substantial portion of the at least one pharmaceutically active agent to the target location and wherein, optionally, the second layer comprises an inert, slowly dissolvable polymer.

53. (canceled)

54. The method of claim 50, wherein the first layer comprises a readily dissolvable polymer comprising the at least one pharmaceutically active agent.

55. The method of claim 50, further comprising a third layer coupled to the second layer, wherein the first and third layers form external layers of the thin film and the second layer forms an internal layer of the thin film between the external layers and comprises the at least one pharmaceutically active agent.

56. The method of claim 55, wherein at least one of the first and third layers comprise the at least one pharmaceutically active agent and/or non-medicinal agents selected from the group consisting of emollients, mydriatics, lubricants, lipids, and combinations thereof.

57. (canceled)

58. (canceled)

59. (canceled)

60. A drug delivery system comprising a thin film configured for positioning at an ocular administration site of a subject for delivery of at least one pharmaceutically active agent to a target location at or near the administration site.

61. The system of claim 60, comprising:
(a) a bioadhesive first layer for adhering to a mucosal surface at the administration site; and
(b) a second layer coupled to the first layer, wherein at least one of the first layer and the second layer comprises the at least one pharmaceutically active agent and wherein, optionally, the second layer is non-bioadhesive.

62. (canceled)

63. The system of claim 61, wherein the second layer limits dispersion of the at least one pharmaceutically active agent to enable focused delivery of a substantial portion of the at least one pharmaceutically active agent to the target location.

64-120. (canceled)
